(19) United States
(12) Patent Application Publication
(10) Pub. No.: US 2016/0279473 A1
(43) Pub. Date: Sep. 29, 2016

(54) Method of Tracking Health and Performance Statistics of Visitors to a Health Theme Park

(71) Applicant: Derrick Robinson, Spartanburg, SC (US)

(72) Inventor: Derrick Robinson, Spartanburg, SC (US)

(21) Appl. No.: 15/076,923

(22) Filed: Mar. 22, 2016

Related U.S. Application Data
(60) Provisional application No. 62/137,050, filed on Mar. 23, 2015.

(51) Int. Cl. A63B 24/00; A61B 5/00; A61B 5/021

(52) U.S. Cl. CPC A63B 24/0062; A61B 5/021; A61B 5/6801; A61B 5/486; A63B 2024/0068; A61B 2503/12

(57) ABSTRACT

A method of tracking health and performance statistics of visitors to a health theme park is utilized to monitor vital signs and activity performance (VSAP) statistics for visitors participating in a theme park activity. At least one database server manages a plurality of user profiles for visitors to the theme park. The VSAP statistics are measured during the theme park activity with a corresponding wearable health monitoring (WHM) device that is associated with the visitor. The visitor is ranked amongst other visitors based on at least one performance metric that is quantitatively assessed based on the VSAP statistics. A performance rank is generated based on the at least one performance metric. The corresponding WHM device is capable of a plurality of ancillary features that may be selected through the corresponding WHM device. A desired feature is executed with the at least one database server.

The current application claims a priority to the U.S. Provisional Patent application Ser. No. 62/137,050 filed on Mar. 23, 2015.

### FIELD OF THE INVENTION

The present invention relates generally to a method for tracking physical activity and performance statistics. More specifically, the present invention is a method of tracking health and performance statistics of visitors to a health theme park.

### BACKGROUND OF THE INVENTION

An increasingly sedentary lifestyle is one of the leading causes of preventable death. Advancements in technology has resulted in increasing amounts of time spent stationary utilizing electronic devices such as computers, smartphones, and televisions. Time commitments such as work and education also contribute to reduced time available and decreased motivation to exercise. Long term, physical inactivity can contribute to a host of health-related issues including obesity, high cholesterol, high blood pressure, and cardiovascular disease. While simply beginning an exercise regimen can be challenging, maintaining a consistent routine once started can be even more difficult. Improvements in general fitness can often be intangible when first beginning an exercise program and progresses gradually over time as the body becomes acclimated to consistent exercise. Because beginning to exercise consistently can be difficult after a period of inactivity, it is common for new exercisers to become discouraged and quit shortly thereafter. It can be argued that tracking activity as well as performance over an extended period of time can encourage new exercisers to maintain a consistent degree of physical activity. For example, a new exerciser who wishes to lose weight may gain encouragement from tracking his or her weight loss over an extended period of consistent physical activity.

The present invention is a method of tracking health and performance statistics of visitors to a health theme park. The present invention enables an individual visitor to track his or her performance statistics during a theme park activity. Within the context of the present invention, the theme park activity generally relates to a physical activity in which the visitor is able to compete with other visitors. The visitor's performance statistics are compared to the performance statistics of other visitors who are competing or have competed in the theme park activity. The individual visitor is assigned points based on his or her performance in the theme park activity with various metrics taken into account in order to determine the number of points. Metrics that may be considered when allocating points include the difficulty of the theme park activity and the age of the individual visitor. In addition to the individual visitor's performance during the theme park activity, the present invention additionally enables the individual visitor to track various vital signs such as blood pressure and heartrate.

### BRIEF DESCRIPTION OF THE DRAWINGS

FIG. 1A is a flowchart depicting the overall process that is followed by the present invention.

FIG. 1B is a continuation of the flowchart depicting the overall process that is followed by the present invention.

FIG. 2 is a flowchart depicting a secondary process that is followed by the present invention.

FIG. 3 is a flowchart depicting a secondary process that is followed by the present invention.

FIG. 4 is a flowchart depicting a secondary process that is followed by the present invention.

FIG. 5 is a flowchart depicting a secondary process that is followed by the present invention.

FIG. 6 is a flowchart depicting a secondary process that is followed by the present invention.

FIG. 7 is a flowchart depicting a secondary process that is followed by the present invention.

FIG. 8 is a flowchart depicting a secondary process that is followed by the present invention.

FIG. 9 is a flowchart depicting a secondary process that is followed by the present invention.

FIG. 10 is a flowchart depicting a secondary process that is followed by the present invention.

FIG. 11 is a flowchart depicting a secondary process that is followed by the present invention.

FIG. 12 is a flowchart depicting a secondary process that is followed by the present invention.

FIG. 13 is a flowchart depicting a secondary process that is followed by the present invention.

FIG. 14 is a flowchart depicting a secondary process that is followed by the present invention.

FIG. 15 is a flowchart depicting a secondary process that is followed by the present invention.

FIG. 16 is a flowchart depicting a secondary process that is followed by the present invention.

### DETAIL DESCRIPTIONS OF THE INVENTION

All illustrations of the drawings are for the purpose of describing selected versions of the present invention and are not intended to limit the scope of the present invention.

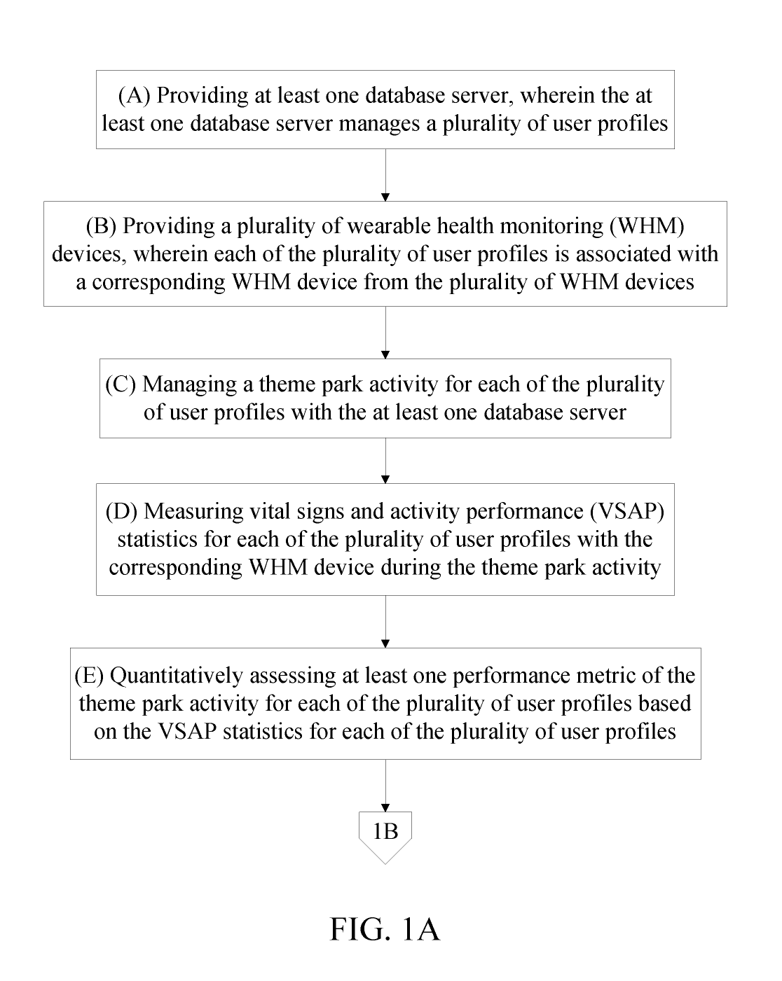

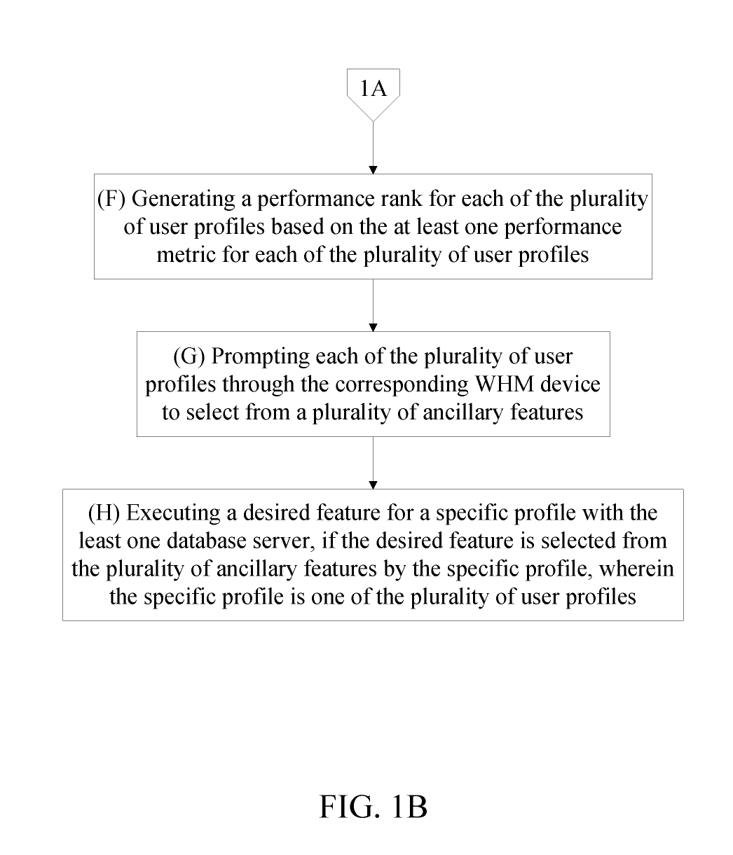

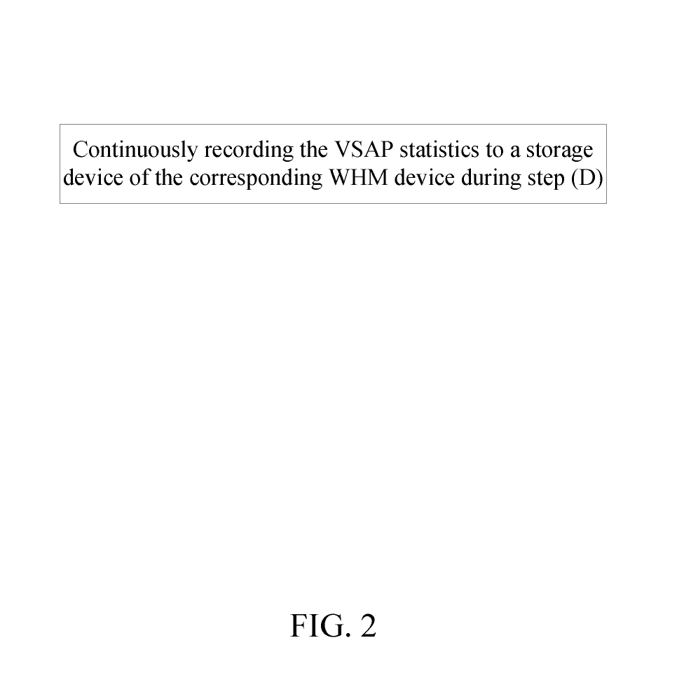

The present invention is a method of tracking health and performance statistics of visitors to a health theme park. The overall process that is followed by the present invention is shown in FIG. 1A and FIG. 1B while secondary processes that are followed by the present invention are shown in FIGS. 2-16.

With reference to FIG. 1A and FIG. 1B, within the context of the present invention, at least one database server is provided. The at least one database server is preferably a storage server that is utilized to store a large amount of data relating to the visitors of the health theme park. The at least one database server manages a plurality of user profiles for the visitors. Each of the plurality of user profiles includes information relating to the visitors including, but not limited to, name, age, gender, height, weight, and similar personal details. A plurality of wearable health monitoring (WHM) devices is assigned to the theme park visitors. Each of the plurality of WHM devices is preferably an electronic wristband or similar device that may easily and conveniently be worn on the user's body. The plurality of WHM devices is preferably durable and waterproof in order to prevent damage incurred during activity. Each of the plurality of user profiles is associated with a corresponding WHM device from the plurality of WHM devices. As such, each visitor to the theme park is assigned a corresponding WHM device that is associated solely with the visitor. The plurality of WHM devices may be utilized to access various amenities and facilities provided by the theme park such as, but not limited to, restaurants, locker rooms, arcades, and transportation services. Additionally, the plurality of WHM devices may include a means for visitors to complete financial transactions directly through the plurality of WHM devices in order to pay for various theme park services. Finally, each of the plurality of WHM devices may be capable of scanning and processing a keychain tag associated with the corresponding user profile from the plurality of user profiles. The keychain tag scanning and processing may be for various applications such as accessing the plurality of user profiles as well as for signing up for theme park activities.

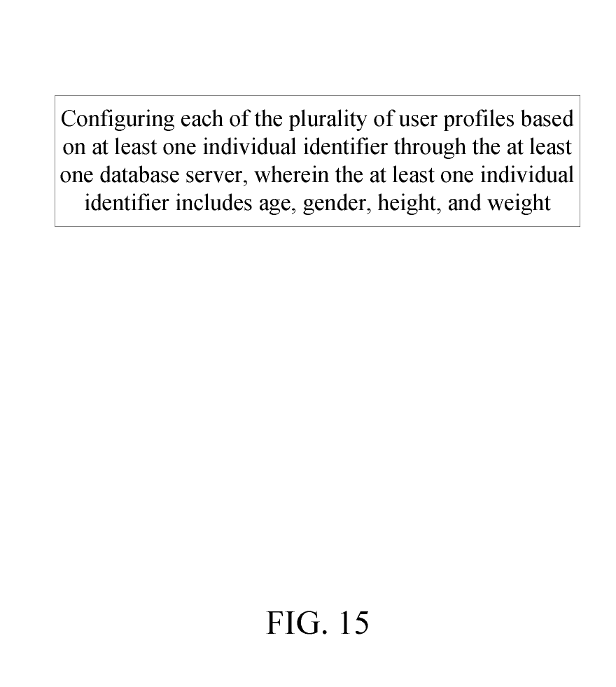

As shown in FIG. 15, each of the plurality of user profiles is configured based on at least one individual identifier through the at least one database server. As such, the visitor is able to provide his or her personal information through the at least one database server. The at least one individual identifier includes age, gender, height, and weight. In the case of height and weight, the visitor may be able to track any changes in his or her height and weight over time through the corresponding WHM device. Visitors may additionally be required to complete a medical consent form and an insurance form as well with all provided information being associated with the plurality of user profiles. The configuration of each of the plurality of user profiles may be completed during the course of registration for membership to the theme park. The visitor may select or be assigned a personal username and password during the configuration process. The username and password may be utilized for various purposes such as accessing personal membership account information for the theme park. A visitor may join a group or organization with other visitors. For example, the visitor may join a group of visitors who attend the same school.

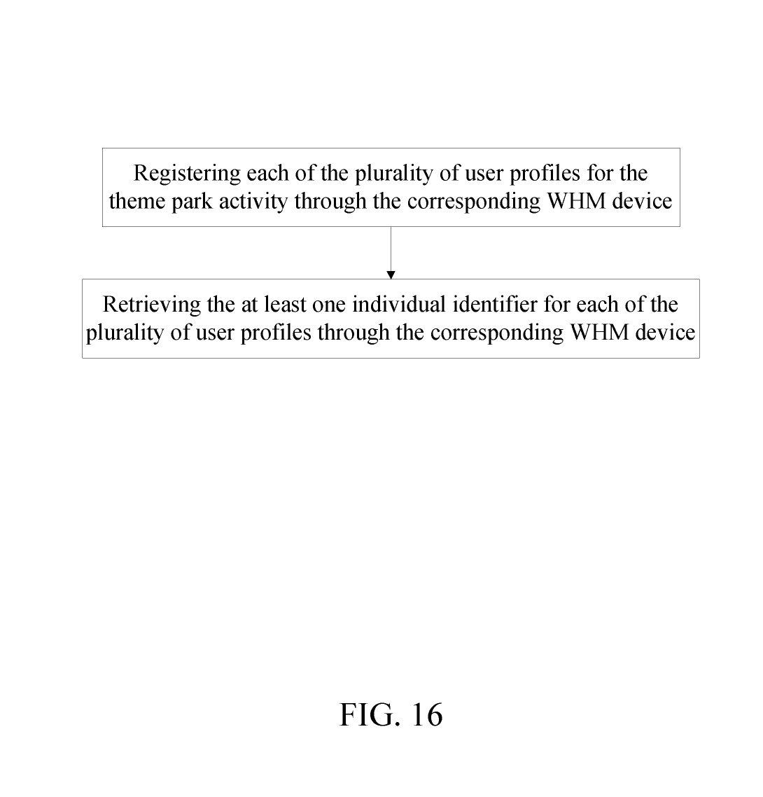

With continued reference to FIG. 1A and FIG. 1B, the theme park as discussed within the context of the present invention includes physically-challenging activities in which visitors are able to participate. Within these activities, visitors may compete against other visitors directly (i.e. competing simultaneously in the same event) or indirectly (i.e. competing separately and receiving an individual score). A theme park activity is managed for each of the plurality of user profiles with the at least one database server. As such, data collected during the course of the theme park activity is managed through the at least one database server. Within the context of the present invention, the theme park activity is a health-related or physical activity-related activity including such as, but not limited to, a race, a game, or another type of athletic competition. As shown in FIG. 16, each of the plurality of user profiles is registered for the theme park activity through the corresponding WHM device. In the preferred embodiment of the present invention, this is accomplished by scanning the corresponding WHM device at a kiosk, booth, or terminal that corresponds to the appropriate theme park activity. A communication protocol such as near field communication (NFC) may be utilized for scanning. In this example, the kiosk, booth, or terminal is connected to the at least one database server. While this is the preferred registration method, registration for the theme park activity may be completed in other ways as well. During registration, the at least one individual identifier for each of the plurality of user profiles is retrieved through the corresponding WHM device. This enables visitors partaking in the theme park activity to be categorized or organized based on the at least one individual identifier if needed.

Again with reference to FIG. 1A and FIG. 1B, in the preferred embodiment of the present invention, vital signs and activity performance (VSAP) statistics for each of the plurality of user profiles are measured for each of the plurality of user profiles with the corresponding WHM device during the theme park activity. More specifically, VSAP statistics are measured and collected for each visitor that is participating in the theme park activity through the corresponding WHM device that is assigned to the visitor. The VSAP statistics may be communicated to the at least one database server via a wireless protocol such as, but not limited to, radio-frequency identification (RFID). Additionally, the VSAP statistics may be continuously recorded to a storage device of the corresponding WHM device as shown in FIG. 2. In this case, the VSAP statistics for each of the plurality of user profiles is stored on the storage device of the corresponding WHM device in addition to the at least one database server.

With continued reference to FIG. 1A and FIG. 1B, at least one performance metric of the theme park activity is quantitatively assessed for each of the plurality of user profiles based on the VSAP statistics for each of the plurality of user profiles. This enables each of the plurality of user profiles to be ranked relative to the remainder of the plurality of user profiles according to the at least one performance metric. A performance rank is generated for each of the plurality of user profiles based on the at least one performance metric for each of the plurality of user profiles. The performance rank is a quantitative indicator of the standing of each of the plurality of user profiles relative to the remainder of the plurality of user profiles with respect to the at least one performance metric.

The plurality of WHM devices includes a number of ancillary features for performing various functions. Each of the plurality of user profiles is prompted through the corresponding WHM device to select from a plurality of ancillary features of which the plurality of WHM devices is capable. As such, a visitor is able to perform a variety of additional functions through the corresponding WHM device in addition to measuring VSAP statistics. A desired feature for a specific profile is executed with the at least one database server if the desired feature is selected from the plurality of ancillary features by the specific profile. In this case, the specific profile is one of the plurality of user profiles belonging to a visitor who wishes to execute the desired feature.

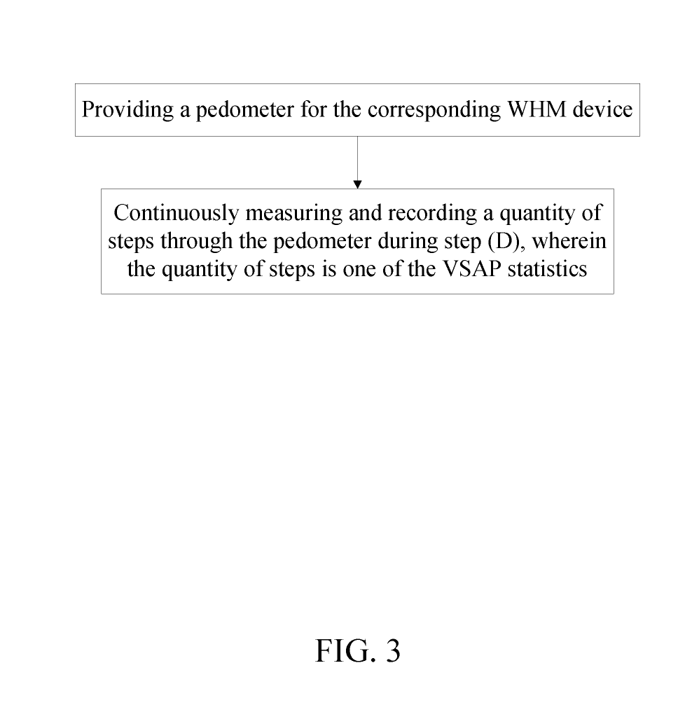

Each of the plurality of WHM devices is capable of measuring a variety of VSAP statistics. As such, each of the plurality of WHM devices comprises hardware that is utilized to measure the VSAP statistics during the course of the theme park activity. As shown in FIG. 3, a pedometer is provided for the corresponding WHM device. While VSAP statistics are being measured with the corresponding WHM device, a quantity of steps is continuously measured and recorded through the pedometer. The quantity of steps is one of the VSAP statistics and may be utilized to calculate additional VSAP statistics such as the total distance traveled by the visitor during the theme park activity. The visitor may keep track of the quantity of steps as the VSAP statistics are aggregated over an extended period of time. The quantity of steps may additionally be utilized to estimate a total number of calories burned as well.

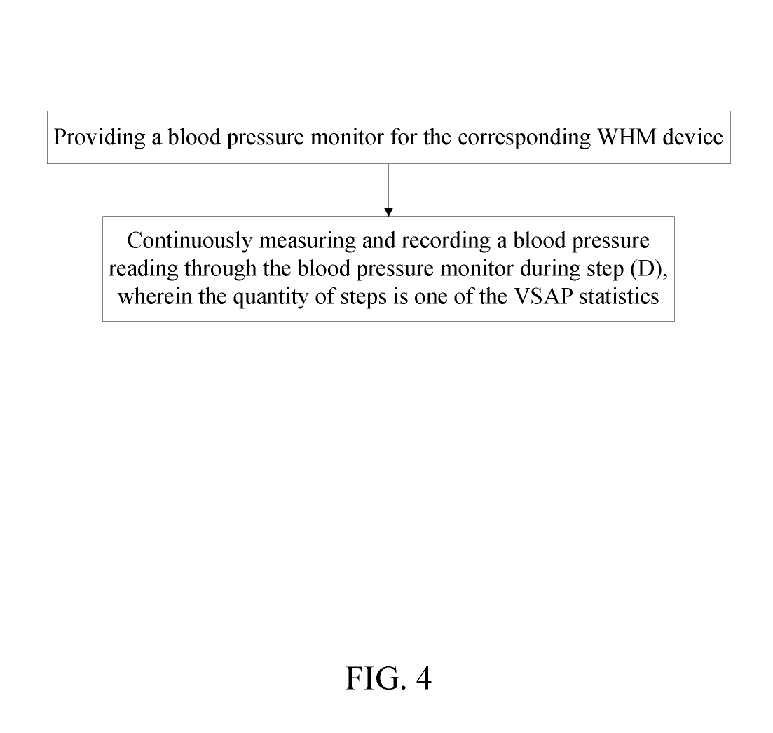

With reference to FIG. 4, a blood pressure monitor is provided for the corresponding WHM device. A blood pressure reading is continuously measured and recorded through the blood pressure monitor while VSAP statistics are being measured with the corresponding WHM device. The blood pressure reading is one of the VSAP statistics and serves as an indicator of one of the most significant vital signs. As with the quantity of steps, the visitor may keep track of the blood pressure reading as the VSAP statistics are aggregated over an extended period of time.

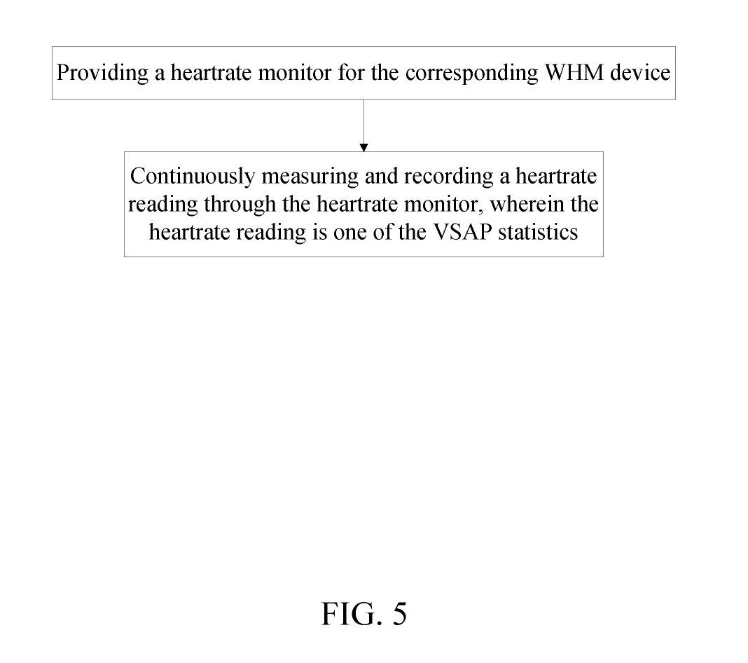

Referring to FIG. 5, a heartrate monitor is provided for the corresponding WHM device. A heartrate reading is continuously measured and recorded through the heartrate monitor while VSAP statistics are being measured with the corresponding WHM device. The heartrate reading is one of the VSAP statistics and much like the blood pressure reading, serves as an indicator for one of the most significant vital signs. The visitor is able to keep track of the heartrate reading as the VSAP statistics are aggregated over an extended period of time.

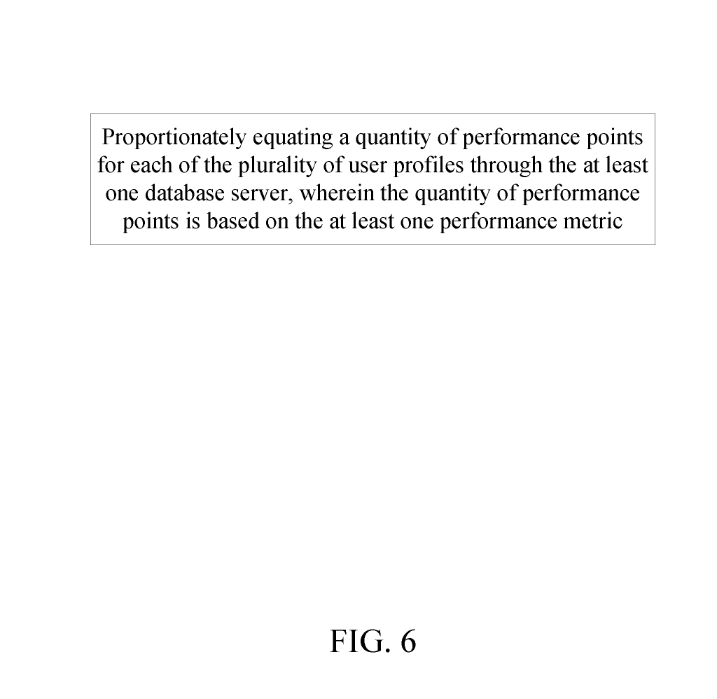

With reference to FIG. 6, a quantity of performance points is proportionately equated for each of the plurality of user profiles through the at least one database server. The quantity of performance points serves as a quantifiable performance-based reward for the visitor following the completion of the theme park activity. Within the context of the present invention, the quantity of performance points may be redeemed for prizes offered by the theme park. The quantity of performance points is based on the at least one performance metric. As such, the at least one performance metric is utilized in order to determine the quantifiable value of the quantity of performance points that is awarded to the visitor. The visitor may track the total quantity of performance points earned over time through the corresponding WHM device.

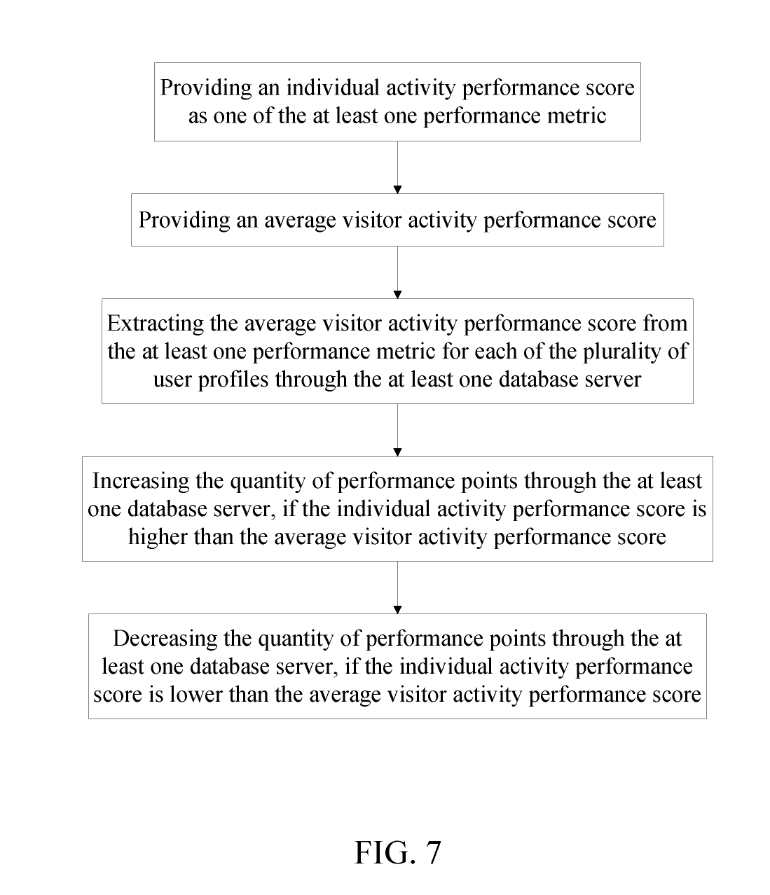

An individual activity performance score may be utilized as one of the at least one performance metric as shown in FIG. 7. In this case, the individual activity performance score is a quantifiable metric for the visitor's performance in the theme park activity and may vary based on the nature of the particular theme park activity. For example, within the context of a race, the individual activity performance score may be a completion time for the race and an associated placement within the standings for all visitors that completed the race. Within the context of a score-based game such as basketball, the individual activity performance score may be a total number of points scored. An average visitor activity performance score is utilized as a metric in order to gauge the individual activity performance score relative to the performance of other visitors. The average visitor activity performance score is extracted from the at least one performance metric for each of the plurality of user profiles through the at least one database server. This ensures that the average visitor activity performance score takes into account the at least one performance metric for all visitors participating in the theme park activity. The quantity of performance points is increased through the at least one database server if the individual activity performance score is higher than the average visitor activity performance score as this is indicative of a stronger than average performance. The quantity of performance points is decreased if the individual activity performance score is lower than the average visitor activity performance score. In this case, a weaker than average performance is awarded a reduced quantity of performance points.

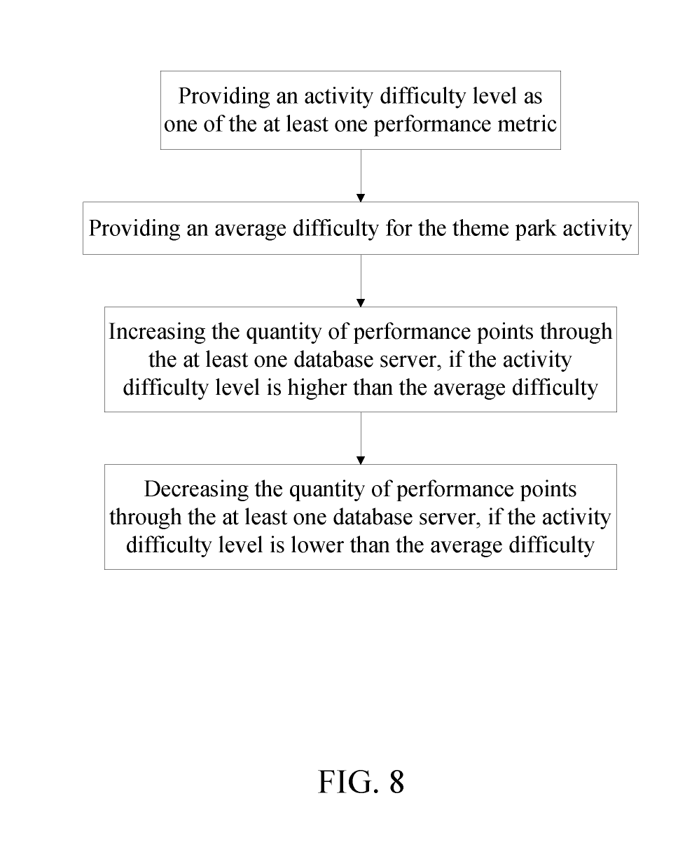

In addition to accounting for visitor performance through the individual activity performance score and the average visitor activity performance score, an activity difficulty level may be utilized as one of the at least one performance metric as shown in FIG. 8. Because of the wide variety of theme park activities that may be available, the difficulty level for the activities may greatly vary as well. In this case, an average difficulty for the theme park activity is utilized. This average difficulty refers to the average difficulty for all theme park activities within the theme park and is utilized to gauge the activity difficulty level relative to the average difficulty. The quantity of performance points is increased through the at least one database server if the activity difficulty level is higher than the average difficulty. This ensures that the visitor is awarded accordingly for participating in a difficult theme park activity. Conversely, the quantity of performance points is decreased through the at least one database server if the activity difficulty level is lower than the average difficulty. As such, the visitor is awarded a reduced quantity of performance points for participating in an easier theme park activity.

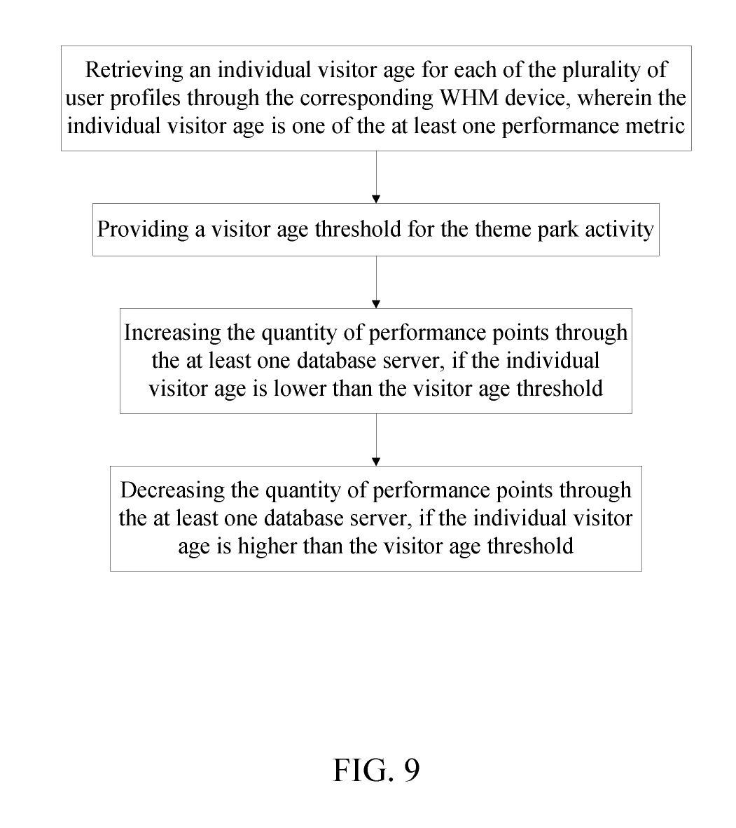

As shown in FIG. 9, an individual visitor age may be utilized as one of the at least one performance metric in order to take age-based performance into consideration during the theme park activity. An individual visitor age is retrieved for each of the plurality of user profiles through the corresponding WHM device. A visitor age threshold for the theme park activity is provided in order to gauge the individual visitor age with respect to the visitor age threshold when utilizing the individual visitor age as one of the at least one performance metric. The following example is discussed with regards to a theme park activity that is designed to be more challenging for children and less challenging for adults. The quantity of performance points is increased through the at least one database server if the individual visitor age is lower than the visitor age threshold. In this case, children as well as other younger visitors are awarded an increased quantity of performance points. Conversely, the quantity of performance points is decreased through the at least one database server if the individual visitor age is higher than the visitor age threshold. As such, adults and older visitors are awarded a decreased quantity of performance points.

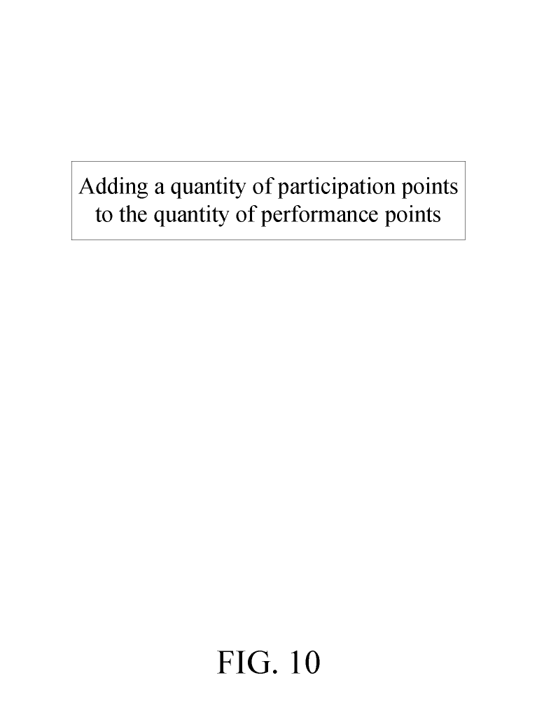

In addition to the quantity of performance points, theme park visitors may be rewarded for simply participating in a theme park activity as shown in FIG. 10. In this case, a quantity of participation points is added to the quantity of performance points. The quantity of participation points may serve as an incentive for visitors to actively participate in theme park activities in order to improve physical fitness and overall health. Much like the quantity of performance points, the quantity of participation points may be redeemed for prizes offered by the theme park.

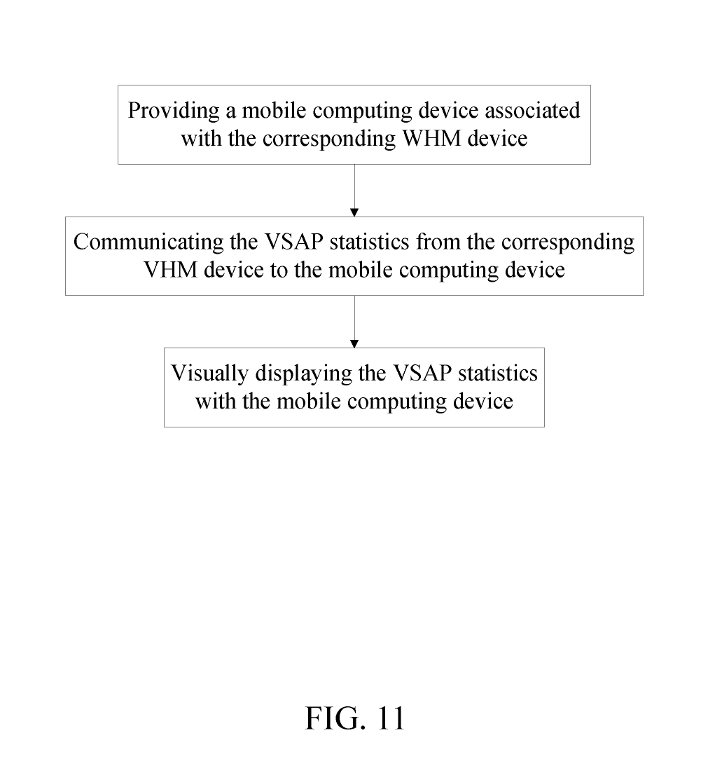

With reference to FIG. 11, the present invention enables a visitor to utilize a mobile computing device such as a smartphone or tablet computer that is associated with the corresponding WHM device. The mobile computing device may be synchronized with the corresponding WHM device in order to enable the two devices to function in conjunction with each other. The VSAP statistics are communicated from the corresponding WHM device to the mobile computing device, allowing the visitor to monitor the VSAP statistics through the mobile computing device. In the preferred embodiment of the present invention, this may be accomplished via a software application that is installed onto the mobile computing device. The VSAP statistics are visually displayed on the mobile computing device in order to allow the user to monitor the VSAP statistics through the mobile computing device in the same manner as through the corresponding WHM device.

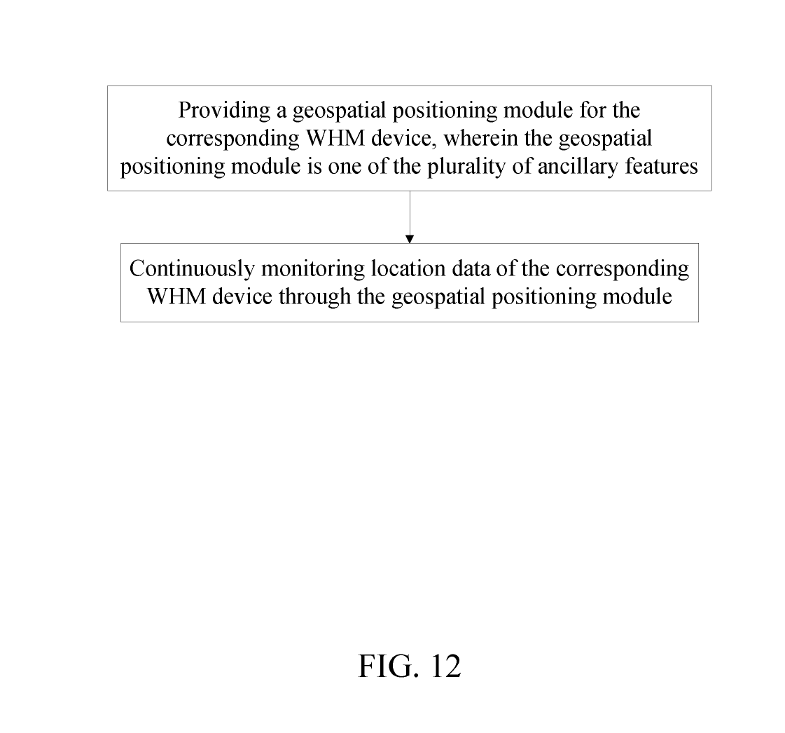

The corresponding WHM device may be utilized to track visitor location for applications such as locating a lost child. As shown in FIG. 12, a geospatial positioning module is provided for the corresponding WHM device. The geospatial positioning module is capable of monitoring the real-time location of the corresponding device and is one of the plurality of ancillary features. Location data of the corresponding WHM device is continuously monitored through the geospatial positioning module. As such, the geospatial positioning module enables the corresponding WHM device to be quickly located in the event that the corresponding WHM device or the visitor with whom the corresponding WHM device is associated is lost.

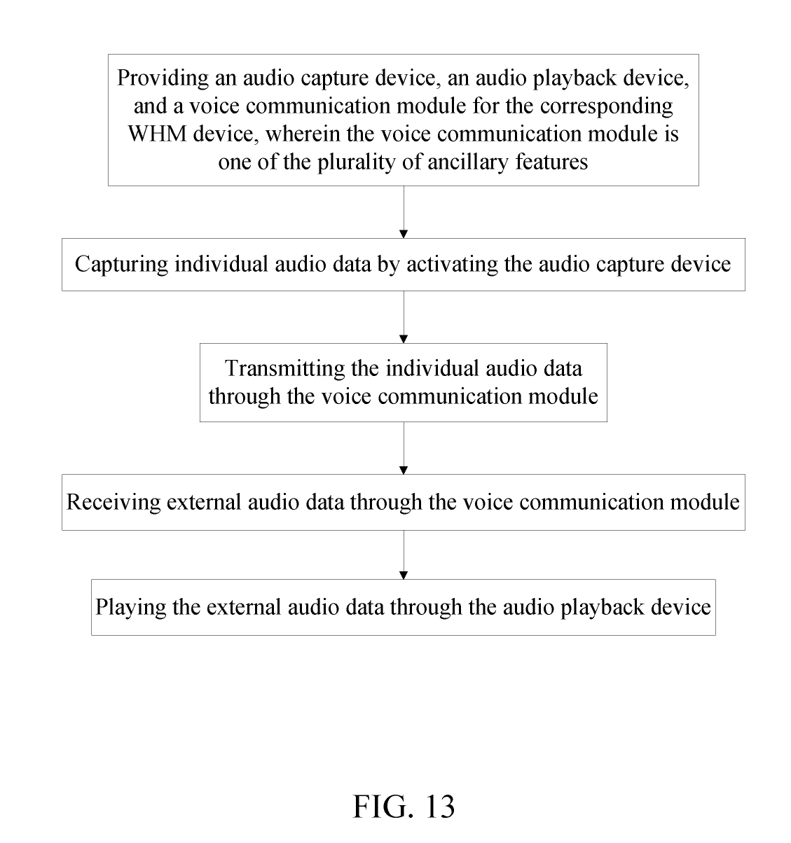

In addition to providing location tracking, the corresponding WHM device may be utilized for wireless communication as shown in FIG. 13. An audio capture device, an audio playback device, and a voice communication module are provided for the corresponding WHM device. The voice communication module is one of the plurality of ancillary features that enables the visitor to communicate with an outside party through the corresponding WHM device. Individual audio data such as the visitor's voice is captured by activating the audio capture device. The audio capture device is preferably a microphone or similar device. The individual audio data is transmitted through the voice communication module and as such may be received by the outside party. Similarly, external audio data from the outside party is received through the voice communication module, enabling two-way communication through the corresponding WHM device. The voice communication module is preferably a wireless transceiver or similar device enabling two-way communication. The external audio data is played through the audio playback device, allowing the visitor to listen to the external audio data from the outside party. The audio playback device is preferably a speaker or similar device.

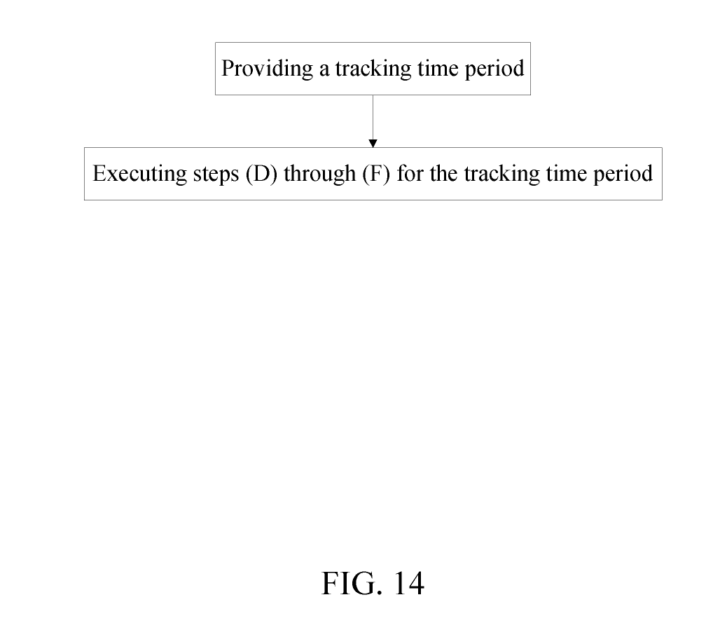

The VSAP statistics for each of the plurality of user profiles may be measured for a provided tracking time period as shown in FIG. 14. The tracking time period is the duration of time for which the VSAP statistics are measured as well as the duration of time for which the plurality of user profiles is ranked with respect to each other based on the at least one performance metric. The at least one performance metric is quantitatively assessed for each of the plurality of user profiles based on the VSAP statistics measured for the tracking time period. The tracking time period may vary in length and may be, but is not limited to, a day, a week, a month, and a year.

While several examples of the plurality of ancillary features of which the plurality of WHM devices is capable have been described, the each of the plurality of WHM devices may be capable of various other functions as well. For example, the plurality of WHM devices may be utilized as hotel room keys or locker keys for visitors to the theme park. In this example, the plurality of WHM devices is able to wirelessly interact with the lock of a hotel room or locker.

Although the present invention has been explained in relation to its preferred embodiment, it is understood that many other possible modifications and variations can be made without departing from the spirit and scope of the present invention as hereinafter claimed.

What is claimed is:

1. A method of tracking health and performance statistics of visitors to a health theme park, the method comprises the steps of:
(A) providing at least one database server, wherein the at least one database server manages a plurality of user profiles;
(B) providing a plurality of wearable health monitoring (WHM) devices, wherein each of the plurality of user profiles is associated with a corresponding WHM device from the plurality of WHM devices;
(C) managing a theme park activity for each of the plurality of user profiles with the at least one database server;
(D) measuring vital signs and activity performance (VSAP) statistics for each of the plurality of user profiles with the corresponding WHM device during the theme park activity;
(E) quantitatively assessing at least one performance metric of the theme park activity for each of the plurality of user profiles based on the VSAP statistics for each of the plurality of user profiles;
(F) generating a performance rank for each of the plurality of user profiles based on the at least one performance metric for each of the plurality of user profiles;
(G) prompting each of the plurality of user profiles through the corresponding WHM device to select from a plurality of ancillary features; and
(H) executing a desired feature for a specific profile with the at least one database server, if the desired feature is selected from the plurality of ancillary features by the specific profile, wherein the specific profile is one of the plurality of user profiles.

2. The method of tracking health and performance statistics of visitors to a health theme park, the method as claimed in claim 1 comprises the steps of:
continuously recording the VSAP statistics to a storage device of the corresponding WHM device during step (D).

3. The method of tracking health and performance statistics of visitors to a health theme park, the method as claimed in claim 1 comprises the steps of:
providing a pedometer for the corresponding WHM device; and
continuously measuring and recording a quantity of steps through the pedometer during step (D), wherein the quantity of steps is one of the VSAP statistics.

4. The method of tracking health and performance statistics of visitors to a health theme park, the method as claimed in claim 1 comprises the steps of:
providing a blood pressure monitor for the corresponding WHM device; and
continuously measuring and recording a blood pressure reading through the blood pressure monitor during step (D), wherein the blood pressure reading is one of the VSAP statistics.

5. The method of tracking health and performance statistics of visitors to a health theme park, the method as claimed in claim 1 comprises the steps of:
providing a heartrate monitor for the corresponding WHM device; and
continuously measuring and recording a heartrate reading through the heartrate monitor, wherein the heartrate reading is one of the VSAP statistics.

6. The method of tracking health and performance statistics of visitors to a health theme park, the method as claimed in claim 1 comprises the steps of:
proportionately equating a quantity of performance points for each of the plurality of user profiles through the at least one database server, wherein the quantity of performance points is based on the at least one performance metric.

7. The method of tracking health and performance statistics of visitors to a health theme park, the method as claimed in claim 6 comprises the steps of:
providing an individual activity performance score as one of the at least one performance metric;
providing an average visitor activity performance score;
extracting the average visitor activity performance score from the at least one performance metric for each of the plurality of user profiles through the at least one database server;
increasing the quantity of performance points through the at least one database server,
if the individual activity performance score is higher than the average visitor activity performance score; and
decreasing the quantity of performance points through the at least one database server,
if the individual activity performance score is lower than the average visitor activity performance score.

8. The method of tracking health and performance statistics of visitors to a health theme park, the method as claimed in claim 6 comprises the steps of:
providing an activity difficulty level as one of the at least one performance metric;
providing an average difficulty for the theme park activity;
increasing the quantity of performance points through the at least one database server,
if the activity difficulty level is higher than the average difficulty; and
decreasing the quantity of performance points through the at least one database server,
if the activity difficulty level is lower than the average difficulty.

9. The method of tracking health and performance statistics of visitors to a health theme park, the method as claimed in claim 6 comprises the steps of:
retrieving an individual visitor age for each of the plurality of user profiles through the corresponding WHM device, wherein the individual visitor age is one of the at least one performance metric;
providing a visitor age threshold for the theme park activity;
increasing the quantity of performance points through the at least one database server,
if the individual visitor age is lower than the visitor age threshold; and
decreasing the quantity of performance points through the at least one database server,
if the individual visitor age is higher than the visitor age threshold.

10. The method of tracking health and performance statistics of visitors to a health theme park, the method as claimed in claim 6 comprises the steps of:
adding a quantity of participation points to the quantity of performance points.

11. The method of tracking health and performance statistics of visitors to a health theme park, the method as claimed in claim 1 comprises the steps of:
providing a mobile computing device associated with the corresponding WHM device;
communicating the VSAP statistics from the corresponding WHM device to the mobile computing device; and
visually displaying the VSAP statistics with the mobile computing device.

12. The method of tracking health and performance statistics of visitors to a health theme park, the method as claimed in claim 1 comprises the steps of:
providing a geospatial positioning module for the corresponding WHM device, wherein the geospatial positioning module is one of the plurality of ancillary features; and
continuously monitoring location data of the corresponding WHM device through the geospatial positioning module.

13. The method of tracking health and performance statistics of visitors to a health theme park, the method as claimed in claim 1 comprises the steps of:
providing an audio capture device, an audio playback device, and a voice communication module for the corresponding WHM device, wherein the voice communication module is one of the plurality of ancillary features;
capturing individual audio data by activating the audio capture device;
transmitting the individual audio data through the voice communication module;
receiving external audio data through the voice communication module; and
playing the external audio data through the audio playback device.

14. The method of tracking health and performance statistics of visitors to a health theme park, the method as claimed in claim 1 comprises the steps of:
providing a tracking time period; and
executing steps (D) through (F) for the tracking time period.

15. The method of tracking health and performance statistics of visitors to a health theme park, the method as claimed in claim 1 comprises the steps of:
configuring each of the plurality of user profiles based on at least one individual identifier through the at least one database server, wherein the at least one individual identifier includes age, gender, height, and weight.

16. The method of tracking health and performance statistics of visitors to a health theme park, the method as claimed in claim 1 comprises the steps of:
registering each of the plurality of user profiles for the theme park activity through the corresponding WHM device; and
retrieving the at least one individual identifier for each of the plurality of user profiles through the corresponding WHM device.
